(19) United States
(12) Patent Application Publication
(10) Pub. No.: US 2022/0072325 A1
(43) Pub. Date: Mar. 10, 2022

(54) HANDPIECE, RF TREATMENT DEVICE, AND RF TREATMENT DEVICE CONTROL METHOD

(71) Applicant: LUTRONIC CORPORATION, Goyang (KR)

(72) Inventor: Kwang Chon KO, Paju (KR)

(21) Appl. No.: 17/416,316

(22) PCT Filed: Dec. 6, 2019
(86) PCT No.: PCT/KR2019/017170

§ 371 Date: Jun. 18, 2021

(30) Foreign Application Priority Data
Dec. 20, 2018 (KR) 10-2018-0166031

(51) Int. Cl. A61N 1/40; A61H 15/02; A61H 15/00

(52) U.S. Cl. CPC A61N 1/40; A61H 15/02; A61H 15/0092; A61N 2001/083; A61H 2201/1695; A61H 2201/5084; A61H 2015/0014

(57) ABSTRACT

An RF treatment device according to an embodiment of the present invention comprises a body having an RF generator, and a handpiece connected to the body, wherein the handpiece: includes a rolling member having a plurality of needles formed to protrude, and a body part, which rotatably supports the rolling member while at least some of the plurality of needles are exposed, which transmits, to the rolling member, RF energy transmitted from the RF generator, and which is formed so that the RF energy is transmitted to the rolling member even while the rolling member is rotating; and further includes a speed measurement unit for detecting information related to the speed of the rolling member, and an RF control unit for controlling the output of the RF energy on the basis of information detected by the speed measurement unit.

### TECHNICAL FIELD

The present disclosure relates to a handpiece, an RF treatment device, and a method of controlling the RF treatment device and, more particularly, to an RF treatment device that is inserted into the tissue of a human body to perform treatment through an invasive method, and a method of controlling the RF treatment device.

### BACKGROUND ART

A method of treating tissue may be classified into a method of treating tissue from outside the tissue, and an invasive treatment method in which a part or all of a treatment device is inserted into the tissue to perform treatment. Among them, the invasive treatment method usually uses a treatment device having an insert part of a small diameter, such as a needle or a catheter, and treatment is performed after the treatment device is inserted to a target location in the tissue.

Such an invasive treatment method includes various treatment procedures, for example, a procedure of delivering a therapeutic substance into tissue, a procedure of performing surgical treatment through the mechanical operation of the treatment device while it is adjacent to specific tissue in the tissue, and a procedure of delivering energy to a target location in the tissue.

As the invasive treatment method, there is an RF treatment method in which a part of all of an RF electrode is inserted into tissue to deliver RF energy thereto.

### DISCLOSURE

### Technical Problem

The present disclosure is to provide a handpiece, an RF treatment device, and a method of controlling the RF treatment device, capable of performing appropriate RF treatment even if the moving speed of the handpiece varies depending on a user.

Technical objects to be achieved by the present disclosure are not limited to the aforementioned technical objects, and other technical objects not described above may be evidently understood by a person having ordinary skill in the art to which the present disclosure pertains from the following description.

### Technical Solution

In order to solve the aforementioned problem, the present disclosure proposes an RF treatment device, including a body having an RF generator, and a handpiece connected to the body, wherein the handpiece includes a rolling member having a plurality of needles formed to protrude, and a body part rotatably supporting the rolling member while at least some of the plurality of needles are exposed, transmitting, to the rolling member, RF energy transmitted from the RF generator, and formed so that the RF energy is transmitted to the rolling member even while the rolling member is rotating, wherein the RF treatment device further includes a speed measurement unit for detecting information related to the speed of the rolling member; and an RF control unit for controlling the output of the RF energy on the basis of information detected by the speed measurement unit.

In order to solve the aforementioned problem, the present disclosure proposes a handpiece which is connected to a body having an RF generator, the handpiece including a rolling member having a plurality of needles formed to protrude; a body part rotatably supporting the rolling member while at least some of the plurality of needles are exposed, transmitting, to the rolling member, RF energy transmitted from the RF generator, and formed so that the RF energy is transmitted to the rolling member even while the rolling member is rotating; and a speed measurement unit for detecting information related to a speed of the rolling member.

In order to solve the aforementioned problem, the present disclosure proposes a method of controlling an RF treatment device including a body having an RF generator, and a handpiece having a rolling member having a plurality of needles formed to protrude, the method including detecting information related to a speed of the rolling member that slides on a surface of skin; and controlling output of the RF energy transmitted to the plurality of needles from the RF generator on the basis of the detected information.

Other specific details of the present disclosure are included in the detailed description and drawings.

### Advantageous Effects

Embodiments of the present disclosure at least have the following effects.

Even if the moving speed of a handpiece varies depending on a user, it is possible to perform appropriate RF treatment.

Further, it is possible to prevent RF energy from being transmitted to a needle that is not inserted into skin but is exposed, thus preventing an accident.

Effects obtained by the present disclosure are not limited to the aforementioned effects, and various other effects are involved in the present disclosure.

### DESCRIPTION OF DRAWINGS

FIG. 1 is a perspective view illustrating an RF treatment device in accordance with an embodiment of the present disclosure.

FIG. 2 is a plan view illustrating a handpiece of FIG. 1.

FIG. 3 is a diagram illustrating an RF treatment method using the handpiece of FIG. 1.

FIG. 4 is a block diagram illustrating the control of the RF treatment device in accordance with an embodiment of the present disclosure.

FIG. 5 is a flowchart illustrating a method of controlling an RF treatment device in accordance with an embodiment of the present disclosure.

### BEST MODE

The above and other objectives, features, and other advantages of the present disclosure will be more clearly understood from the following detailed description when taken in conjunction with the accompanying drawings. However, the disclosure may be embodied in different forms without being limited to the embodiments set forth herein. Rather, the embodiments disclosed herein are provided to make the disclosure thorough and complete and to sufficiently convey the spirit of the present disclosure to those skilled in the art. The present disclosure is to be defined by the claims.

Further, embodiments set forth herein will be described with reference to sectional views and/or schematic views that are ideal exemplary views of the present disclosure. Thus, the exemplary views may be modified by manufacturing technology and/or tolerance. Furthermore, the size or shape of components shown in the drawings may be exaggerated for the clarity and convenience of description. The same reference numerals are used throughout the drawings to designate the same or similar components.

Hereinafter, the term “RF treatment device” covers all devices for treating humans as well as animals such as mammals. The treatment device may include various devices that transmit RF energy for the purpose of improving the condition of a lesion or tissue. Although a device for treating a skin lesion will be mainly described in the following embodiment, the present disclosure is not limited thereto. That is, it is to be understood that the treatment device may be applied to various devices that transmit RF energy to various affected areas, including a device for surgically treating internal organ lesions.

Hereinafter, the term “tissue” refers to the assembly of cells constituting various body organs of animals including humans, and covers various tissues forming various internal organs as well as skin tissue.

Hereinafter, the term “treatment” refers to remodeling in which RF energy is transmitted to tissue including collagen to change the state of the tissue into at least one of coagulation or ablation, and may be treatment for at least one of wrinkles, tone and textural changes, scars and acne scarring, sagging mucosa, overall rejuvenation, hyperhidrosis, laxity, lifting, tightening, and fat reduction for skin tissue, for example.

Hereinafter, an RF treatment device and a control method of the RF treatment device according to an embodiment of the present disclosure will be described with reference to the drawings.

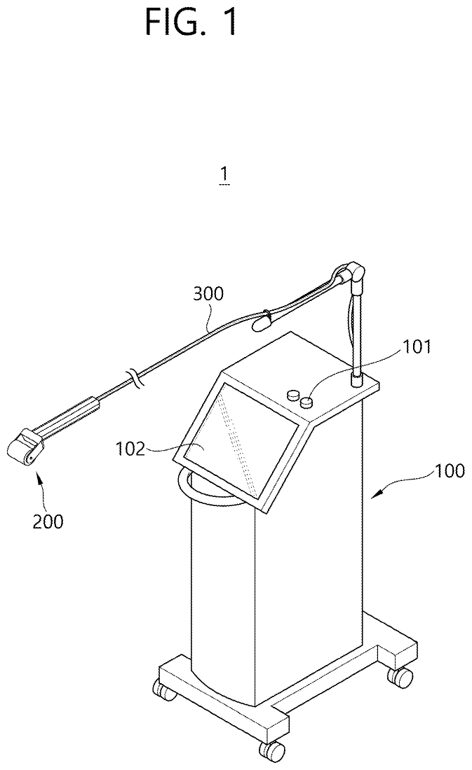

FIG. 1 is a perspective view illustrating an RF treatment device in accordance with an embodiment of the present disclosure.

As shown in FIG. 1, the RF treatment device 1 according to this embodiment includes a body 100 and a handpiece 200 that is held by a user to perform treatment.

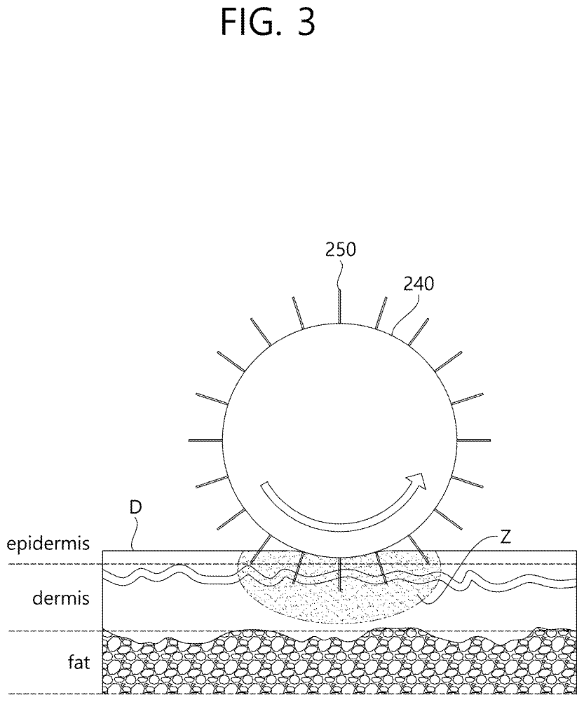

An RF generator 111 (see FIG. 3) may be provided in the body 100. The RF generator 111 generates RF energy used for treatment. The RF generator 111 is configured to generate and transmit the RF energy not in a continuous waveform but in a pulse form. The RF generator 111 may generate RF pulses of various parameters (e.g. output, pulse duration, pulse interval, frequency, etc.) according to a patient's constitution, a treatment purpose, or a treatment site. The RF pulse generated in the RF generator of this embodiment is a therapeutic RF pulse used for the purpose of treating tissue. The RF energy used for treating the skin may be adjusted in the range of 0.1 to 0.8 MHz.

A switch 101 for adjusting the operation of the treatment device, e.g., the operation of turning on or off a power supply, and a display unit 102 for displaying various pieces of information including the operation of the treatment device may be included on the outer surface of the body 100. Such a display unit 102 may be formed of a touch screen to display various pieces of information, and may be configured such that a user may directly set treatment contents through the display unit 102.

The handpiece 200 is connected to the body via a connector 300. The connector 300 may transmit power or a control signal required to operate various devices of the handpiece 200 from the body 100. Furthermore, the connector 300 may transmit the RF energy from the RF generator 111 to the handpiece 200. To this end, the connector 300 may be formed of a cable including various signal lines and power lines, or may have a bent structure to be easily bent by a user's manipulation.

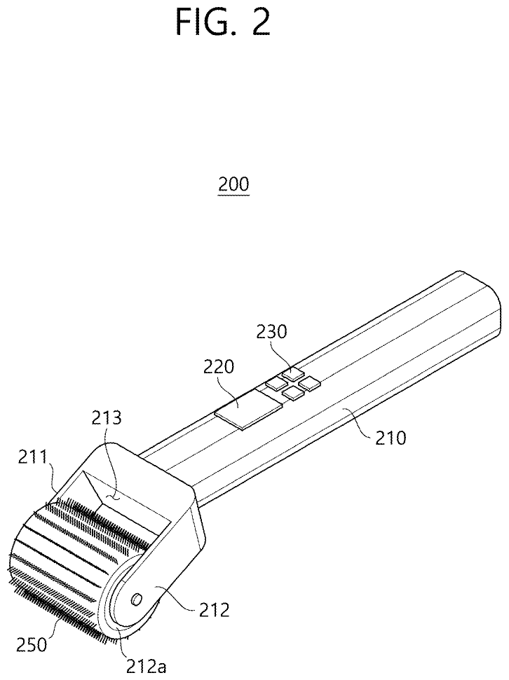

FIG. 2 is a plan view illustrating the handpiece of FIG. 1.

As shown in FIG. 2, the handpiece 200 includes a body part 210 and a rolling member 240.

A handpiece operating part 230 and a handpiece display part 220 may be provided on an outer surface of the body part 210. The handpiece operating part 230 may be configured to control the on/off of the handpiece 200 or the intensity of the RF energy transmitted through the needle 250. The display unit 220 of the handpiece may display various pieces of information required in a setting mode or during treatment to a user. Thus, a user may perform treatment by operating the operating part 230 with the handpiece 200 being held by his or her hand, and simultaneously check treatment contents easily through the display unit 220.

The above-described connector 300 is connected to a rear end of the body part 210, and the rolling member 240 is coupled to a front end of the body part 210. To this end, as shown in FIG. 2, a pair of support arms 211 and 212 extends from the front end of the body part 210 while being spaced apart from each other, and defines a rolling-member accommodation space 213.

The first support arm 211 rotatably supports one side of the rolling member 240, while the second support arm 212 rotatably supports the other side of the rolling member 240. The first support arm 211 and the second support arm 212 support a horizontal central axis (not shown) of the rolling member 240 to allow the rolling member 240 to be stably rotated.

A signal line and a power line electrically connected to the rolling member 240 may be provided in the first support arm 211 and/or the second support arm 212 so that the RF energy transmitted through the connector 300 and the body 100 may be transmitted to the rolling member 240 through at least one of the first support arm 211 and the second support arm 212, and information collected from the rolling member 240 is transmitted through at least one of the first support arm 211 and the second support arm 212 to the body 100.

At least one of the first support arm 211 and the second support arm 212 includes a coupling element 212a.

The coupling element 212a serves to maintain electrical connection with the signal line and the power line of the first support arm 211 and/or second support arm 212 even when the rolling member 240 is rotating. To this end, the coupling element 212a may include a brush element that maintains the electrical connection of a stationary part and a movable part.

As shown in FIG. 2, the rolling member 240 may substantially have a cylindrical shape, and a plurality of needles 250 may be formed on a curved surface corresponding to a side of the cylinder to protrude therefrom. The needle 250 of this embodiment may be a needle having a diameter ranging from several micrometers to several thousand micrometers (μm), and preferably may use a needle having a diameter ranging from 10 to 1000 μm.

The plurality of needles 250 may extend radially from the horizontal central axis of the rolling member 240. The plurality of needles 250 may be formed of a conductive material to receive and release the RF energy transmitted through the rolling member 240. A part of a surface of each needle excluding its front end may be formed of an insulating material to prevent the RF energy from being transmitted to the tissue. Thereby, each needle is configured such that a part of the front end thereof serves as an electrode, and the RF energy is transmitted to the tissue only through the front end. Thus, it is possible to selectively transmit the RF energy to a part at which the end of the needle is located during treatment.

The rolling member 240 may be configured such that the plurality of needles 250 is divided into a plurality of needle groups, and RF energy is independently transmitted to each needle group. Each needle group may include a few or tens of needles 250. According to an embodiment, at least some of the needle groups may include one needle 250. According to an embodiment, the needle groups may be divided on the basis of a row that is perpendicular to the forward and/or backward movement direction of the rolling member 240.

To this end, the rolling member 240 may include an electric circuit in which the plurality of needles 250 is divided into the plurality of needle groups and is electrically connected in a matrix form. The electric circuit is electrically connected to the coupling element 212a of the first support arm 211 and/or the second support arm 212 to receive the RF energy transmitted through the first support arm 211 and/or the second support arm 212 to the rolling member 240 and thereby transmit the RF energy to at least some of the plurality of needles 250.

Preferably, the rolling member 240 is manufactured to be separable from the body part 210. In this case, the rolling member 240 is configured to be disposable, so that the rolling member is separated from the body part 210 and then is discarded after the RF treatment is performed for one patient. When the RF treatment is performed on another patient, a new rolling member 240 may be coupled to the body part 210.

FIG. 3 is a diagram illustrating an RF treatment method using the handpiece of FIG. 1.

As shown in FIG. 3, the rolling member 240 slides on a surface of a patient's skin D while rotating. Although not shown in the drawing, the handpiece 200 may move forwards and backwards with the user holding the body part 210 of the handpiece 200, thus allowing the rolling member 240 to be rotated on the surface of the patient's skin D.

As the rolling member 240 rotates while coming into close contact with the surface of the skin D, the needle 250 is sequentially inserted into the skin D and then is removed from the skin D. This process is repeated.

The RF energy is released from the front end of the needle 250 inserted into the skin D, and the RF energy is transmitted to the interior of the tissue at a treatment location Z, so that treatment is performed. Since a location where the needle 250 is inserted varies as the rolling member 240 moves forwards or backwards, the treatment location Z is correspondingly moved.

Although FIG. 3 illustrates an example in which the needle 250 is inserted into the dermal layer of the skin D, the length of the needle 250 may vary depending on an RF treatment site. For example, when the RF treatment is performed on the epidermal layer of the skin D, the rolling member on which needles of shorter lengths are formed may be used as compared to the example shown in FIG. 3. When the RF treatment is performed in a lower area of the dermal layer of the skin D, the rolling member on which needles of longer lengths are formed may be used as compared to the example shown in FIG. 3. According to an embodiment, the rolling member on which needles 250 of different lengths are formed may be used. In this case, the RF treatment may be performed throughout the epidermal layer and/or the dermal layer of the skin D.

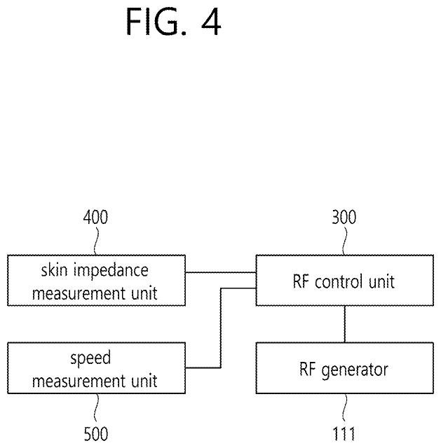

FIG. 4 is a block diagram illustrating the control of the RF treatment device in accordance with an embodiment of the present disclosure.

As shown in FIG. 4, the RF treatment device 1 according to the embodiment of the present disclosure further includes an RF control unit 300, a skin impedance measurement unit 400, and a speed measurement unit 500.

The skin impedance measurement unit 400 is configured to monitor the state information of the tissue corresponding to the treatment location Z during treatment.

The skin impedance measurement unit 400 may be provided on a path to which the RF energy is transmitted, and may be configured to measure the impedance of the path to which the RF energy is transmitted after passing through the tissue. The skin impedance measurement unit 400 may be provided on an RF transfer path in the handpiece 200, and may be provided on an RF transfer path in the body.

The skin impedance measurement unit 400 may monitor an impedance value by causing a separate test current to flow through the plurality of needles 250, and may monitor an impedance value measured while the RF energy is transmitted. Since the measured impedance varies depending on the characteristics of a patient or a change in skin condition, this impedance may be interpreted as ‘skin impedance’ for convenience.

The information measured by the skin impedance measurement unit 400 is information related to the skin impedance. This may be an impedance value of the treatment location Z in the needle 250 is inserted, and may be information for inferring the impedance of the treatment location Z.

Meanwhile, since the information measured by the skin impedance measurement unit 400 is acquired through the needle 250 inserted into the skin, it is possible to confirm the needle 250 inserted into the skin among the plurality of needles 250 on the basis of the measured information. The rolling member 240 may be controlled to receive information about the needle 250 inserted into the skin from the skin impedance measurement unit 400 and to transmit the RF energy to only the needle 250 inserted into the skin. When the plurality of needles 250 is divided into the plurality of needle groups, control may be performed such that the RF energy is transmitted to only the needle group including the needle 250 inserted into the skin.

Thereby, since the RF energy is transmitted to only the needle 250 inserted into the skin D and the RF energy is not transmitted to the needle 250 that is not inserted into the skin D but is exposed, it is possible to prevent a user or a patient from getting unexpected electric shocks caused by the needle 250 that is not inserted into the skin D but is exposed, during treatment.

The speed measurement unit 500 is configured to monitor information related to the moving speed of the handpiece 200 and/or the rolling member 240.

The speed measurement unit 500 may be configured to detect the moving speed of the handpiece 200, or to detect the rotating speed or the moving speed of the rolling member 240. For example, the speed measurement unit 500 may include an acceleration sensor provided in the body part 210 of the handpiece 200 to detect the moving speed of the handpiece 200. Alternatively, an encoder, a tachometer for measuring RPM in an optical manner or a Keyphasor may be provided in the front end of the body part 210 of the handpiece 200 to detect the rotating speed of the rolling member 240. They are merely examples of the speed measurement unit 500, and the present disclosure is not limited thereto. Various configurations capable of detecting the moving speed of the handpiece 200 or detecting the rotating speed or the moving speed of the rolling member 240 may be used as the speed measurement unit 500.

Information measured by the speed measurement unit 500 may be information related to speed (the moving speed of the handpiece 200, the rotating speed of the rolling member 240, and the moving speed of the rolling member 240). This may be a speed value, and may be information for inferring speed.

The RF control unit 300 receives information related to the skin impedance from the skin impedance measurement unit 400, and receives information related to the speed from the speed measurement unit 500. Further, the RF control unit may control the RF generator 111 on the basis of information transmitted from the skin impedance measurement unit 400 and the speed measurement unit 500 to adjust the on/off operation of the RF pulse and the parameter of the RF pulse.

The RF control unit 300 controls the RF generator 111 on the basis of information transmitted from the skin impedance measurement unit 400. Since the skin impedance varies depending on a patient or a body region and a change occurs in skin tissue of the treatment location Z during the RF treatment, the skin impedance is also changed during the RF treatment. Even if the same RF energy is applied, a therapeutic effect realized in the tissue of the treatment location Z varies depending on the impedance of the treatment location Z.

Thus, for optimal RF treatment, RF energy optimized for the impedance of the treatment location Z should be applied. Since the RF control unit 300 receives the information, related to the impedance of the treatment location Z into which the needle 250 is inserted, in real time and controls the output of the RF energy in response to the received information, it is possible to achieve the optimal RF treatment for the treatment location Z.

The RF control unit 300 controls the RF generator 111 on the basis of the information related to the speed from the speed measurement unit 500.

In order to provide appropriate therapeutic effect for the treatment location Z, the RF energy that is equal to or more than a predetermined level should be applied to the tissue of the treatment location Z. However, due to a difference in speed at which the handpiece 200 is operated (the rolling member 240 slides on a skin surface) depending on a user, the handpiece 200 may be operated at a speed that is higher or lower than an expected speed in a step of manufacturing the RF treatment device 1. In this case, sufficient therapeutic effect may not be realized, or RF energy more than necessary may be applied to the treatment location Z, thus possibly causing side effects such as burns.

To this end, the RF control unit 300 may receive the information related to the speed from the speed measurement unit 500, may control the RF generator 111 to increase the output of the RF energy when the received information corresponds to a state in which the speed of the handpiece 200 and/or the rolling member 240 is higher than a reference speed (a preset value), and may control the RF generator 111 to decrease the output of the RF energy when the received information corresponds to a state in which the speed of the handpiece 200 and/or the rolling member 240 is lower than the reference speed.

The RF control unit 300 may control the RF generator 111 so that the increased amount/decreased amount of the output of the RF energy is proportional to a difference between the speed of the handpiece 200 and/or the rolling member 240 and the reference speed.

Thereby, even if a user performs the treatment while rotating the rolling member 240 at a speed higher than that expected by a manufacturer of the RF treatment device 1, sufficient RF energy may be applied to the treatment location Z because the RF control unit 300 increases the output of the RF energy. In contrast, even if a user performs the treatment while rotating the rolling member 240 at a speed lower than that expected by a manufacturer of the RF treatment device 1, excessive RF energy may be prevented from being applied to the treatment location Z because the RF control unit 300 decreases the output of the RF energy.

Although the RF control unit 300 may be provided on the body 100, it may be provided on the handpiece 200 according to an embodiment.

The RF treatment device 1 according to an embodiment of the present disclosure may further include a monitoring unit that monitors the temperature of tissue or monitors at least one of various pieces of information required for treatment, such as the contact of the handpiece or the pressed state. In this case, the RF control unit 300 may receive information monitored from the monitoring unit and may control the RF control unit 300 on the basis of the received information, thus controlling the output of the RF energy.

According to an embodiment, the monitoring unit may include the skin impedance measurement unit 400.

Furthermore, the RF treatment device 1 according to an embodiment of the present disclosure may further include a setting unit that allows a user to set treatment contents. The setting unit may be composed of the above-described display unit 102 and/or a switch, and may perform a setting operation in a manner of displaying various options to a user through the display unit 102 and selecting a displayed option by the user.

Furthermore, the RF treatment device 1 according to an embodiment of the present disclosure may further include a memory unit in which various data is stored, and may perform a control operation by storing information required for controlling the RF treatment device in the memory unit or reading data stored in the memory unit.

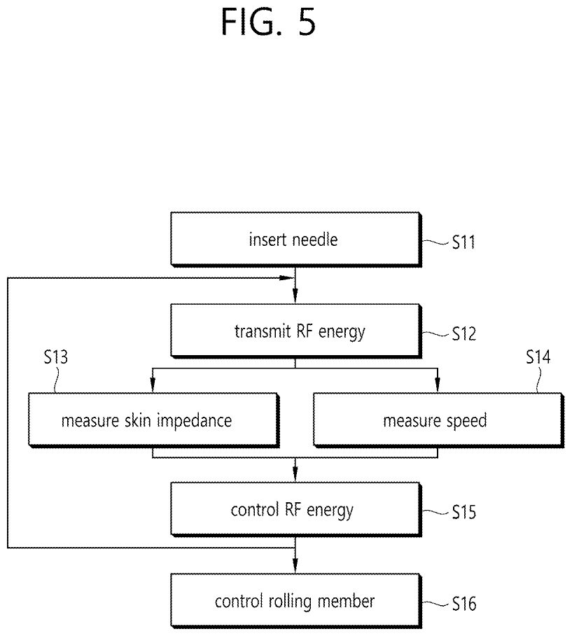

Hereinafter, a method of controlling the RF treatment device according to an embodiment of the present disclosure will be described. FIG. 5 is a flowchart illustrating the method of controlling the RF treatment device in accordance with an embodiment of the present disclosure.

As shown in FIG. 5, the method of controlling the RF treatment device in accordance with the embodiment of the present disclosure may include a step S11 of inserting a needle, a step S12 of transmitting RF energy, a step S13 of measuring skin impedance, a step S14 of measuring a speed, a step S15 of controlling RF energy, and a step S16 of controlling a rolling member.

In the needle inserting step S11, a user holds the handpiece 200, puts the rolling member 240 on a skin surface of a patient's treatment site to come into close contact therewith, and then inserts some of the plurality of needles 250 into the skin D. As soon as the initial needle 250 is inserted into the skin D, the handpiece 200 may be moved to cause the rolling member 240 to slide on the skin surface.

In the RF-energy transmitting step S11, the RF generator 111 generates the RF energy, and the body 100 transmits the generated RF energy through the connector 300 to the handpiece 200. The RF energy transmitted to the handpiece 200 is transmitted through the first support arm 211 and/or the second support arm 212 to the rolling member 240, and the rolling member 240 transmits the RF energy to at least some of the plurality of needles 250.

When the RF energy is first transmitted to the rolling member 240, the rolling member 240 may transmit the RF energy to all of the plurality of needles 250. This may be intended to identify the location of the needle 250 inserted into the skin and to measure the skin impedance. In this case, the needle inserting step S11 may be performed subsequent to the RF-energy transmitting step S12.

In the case of measuring the skin impedance using the RF energy, the RF-energy transmitting step S12 should precede the skin-impedance measuring step S13 that will be described later. However, when the skin impedance measurement unit 400 monitors the impedance value by causing a separate test current to flow through the plurality of needles 250, the RF-energy transmitting step S12 may be performed subsequent to the RF-energy controlling step S15 that will be described later.

In the skin-impedance measuring step S13, the skin impedance measurement unit 400 acquires information related to the impedance of the treatment location Z into which the needle 250 is inserted. Further, the needle 250 inserted into the treatment location Z may be sensed on the basis of several pieces of impedance information measured from the plurality of needles 250.

In the speed-measuring step S14, the speed measurement unit 500 acquires information related to the speed (the moving speed of the handpiece 200, the rotating speed of the rolling member 240, and the moving speed of the rolling member 240).

In the RF-energy controlling step S15, the RF control unit 300 receives information related to the skin impedance from the skin impedance measurement unit 400, and receives information related to the speed from the speed measurement unit 500 to control the RF generator 111 on the basis of the received information, thus adjusting the on/off operation of the RF pulse and the parameter of the RF pulse.

The RF control unit 300 may be configured to receive information from each of the skin impedance measurement unit 400 and the speed measurement unit 500 in real time and thereby control the RF generator 111.

Since specific examples of the configuration where the RF control unit 300 controls the RF generator 111 on the basis of information transmitted from each of the skin impedance measurement unit 400 and the speed measurement unit 500 have been described above, a detailed description thereof will be omitted herein.

The RF generator 111 may generate the RF energy by the configuration controlled in the RF-energy controlling step S15, and the RF-energy transmitting step S12 may be performed again. That is, the RF control unit 300 feedback controls the RF generator 111 in response to the information transmitted from the skin impedance measurement unit 400 and the speed measurement unit 500 in real time, thus allowing the RF energy optimal for treatment to be transmitted to the tissue of the treatment location Z, in response to a change in skin impedance and a change in speed of the handpiece 200 and/or the rolling member 240.

In the rolling-member controlling step S16, the rolling member 240 is controlled to transmit the RF energy to only the needle 250 inserted into the skin, which is identified in the skin-impedance measuring step S13. In the case of including the electric circuit in which the plurality of needles 250 is electrically connected in a matrix form in the rolling member 240, the electric circuit is controlled to transmit the RF energy to only the needle 250 inserted into the skin. In the case of including the electric circuit in which the plurality of needles 250 is divided into a plurality of needle groups and then is electrically connected, the electric circuit is controlled to transmit the RF energy to only a needle group including the needles 250 inserted into the skin.

Thereby, since the RF energy is transmitted to only the needle 250 inserted into the skin D and the RF energy is not transmitted to the needle 250 that is not inserted into the skin D but is exposed, it is possible to prevent a user or a patient from getting unexpected electric shocks caused by the needle 250 that is not inserted into the skin D but is exposed, during treatment.

As a user moves the handpiece 200 forwards or backwards, the needle 250 inserted into the skin D of the patient is changed, and simultaneously the treatment location Z is also changed. Thus, steps S12 to S16 are repeatedly performed to cause the RF energy suitable for the treatment location Z to be transmitted.

As described above, since the RF treatment device 1 and the control method of the RF treatment device according to an embodiment of the present disclosure control the RF energy in response to the speed of the handpiece 200 and/or the rolling member 240, the appropriate RF energy is applied to the tissue of the treatment location Z even if the speed of moving the handpiece 200 varies depending on a user.

Furthermore, in the RF treatment device 1 and the control method of the RF treatment device according to an embodiment of the present disclosure, the RF energy is transmitted to only the needle 250 inserted into the skin D and the RF energy is not transmitted to the needle 250 that is not inserted into the skin D but is exposed, so that it is possible to prevent a user or a patient from getting unexpected electric shocks caused by the needle 250 that is not inserted into the skin D but is exposed, during treatment.

Hereinbefore, the description has been focused on the treatment device that transmits the RF energy to the skin tissue to perform treatment. However, this is merely illustrative, but may be applied to a treatment device for treating tissues other than the skin tissue. Furthermore, the present disclosure may be applied to various treatment devices of performing treatment by transmitting energy such as RF, laser, or ultrasonic waves, as well as the treatment device of performing treatment by transmitting RF energy, and may be applied to various treatment devices of performing treatment by transmitting a therapeutic substance (e.g. medication, anesthetics, stem cells, etc.).

Although the description has focused on the treatment device composed of the body and the handpiece, the present disclosure may be applied to a treatment device formed of a single handpiece module without being limited thereto.

It is evident to those skilled in the art that the present disclosure may be materialized in other specific forms without departing from the essential characteristics of the present disclosure. Accordingly, the detailed description should not be construed as being limitative from all aspects, but should be construed as being illustrative. The scope of the present disclosure should be determined by reasonable analysis of the attached claims, and all changes within the equivalent range of the present disclosure are included in the scope of the present disclosure.

### MODE FOR DISCLOSURE

An RF treatment device according to an embodiment of the present disclosure includes a body having an RF generator, and a handpiece connected to the body. The handpiece includes a rolling member having a plurality of needles formed to protrude, and a body part rotatably supporting the rolling member while at least some of the plurality of needles are exposed, transmitting, to the rolling member, RF energy transmitted from the RF generator, and formed so that the RF energy is transmitted to the rolling member even while the rolling member is rotating, and further includes a speed measurement unit for detecting information related to a speed of the rolling member, and an RF control unit for controlling output of the RF energy on the basis of information detected by the speed measurement unit.

The RF control unit may increase the output of the RF energy when information detected by the speed measurement unit corresponds to a state where the speed of the rolling member is higher than a reference speed.

The RF control unit may decrease the output of the RF energy when information detected by the speed measurement unit corresponds to a state where the speed of the rolling member is lower than the reference speed. [%] The speed of the rolling member may be a rotating speed or a moving speed of the rolling member.

The RF treatment device may further include a skin impedance measurement unit configured to measure impedance of skin on the basis of information about the RF energy transmitted through the needle inserted into the skin among the plurality of needles.

The RF control unit may control the output of the RF energy on the basis of the skin impedance measured by the skin impedance measurement unit.

The RF treatment device may further include a skin impedance measurement unit configured to measure the skin impedance on the basis of information about a current and a voltage transmitted through the needle inserted into the skin among the plurality of needles, the rolling member may be configured such that the plurality of needles is divided into a plurality of needle groups, and the RF energy transmitted through the body part is independently transmitted to each needle group, so that the rolling member may be configured to sense a needle group including the needle inserted into the skin among the plurality of needle groups on the basis of the skin impedance measured by the skin impedance measurement unit, and to transmit the RF energy to the sensed needle group.

A handpiece according to an embodiment of the present disclosure may be a handpiece connected to a body having an RF generator, the handpiece including a rolling member having a plurality of needles formed to protrude, a body part rotatably supporting the rolling member while at least some of the plurality of needles are exposed, transmitting, to the rolling member, RF energy transmitted from the RF generator, and formed so that the RF energy is transmitted to the rolling member even while the rolling member is rotating, and a speed measurement unit for detecting information related to a speed of the rolling member.

The handpiece may further include an RF control unit for controlling output of the RF energy on the basis of information detected by the speed measurement unit.

The speed of the rolling member may be a rotating speed or a moving speed of the rolling member.

The handpiece may further include a skin impedance measurement unit configured to measure the skin impedance on the basis of information about a current and a voltage transmitted through the needle inserted into the skin among the plurality of needles.

The rolling member may be configured such that the plurality of needles is divided into a plurality of needle groups, and the RF energy transmitted through the body part is independently transmitted to each needle group, so that the rolling member may be configured to sense a needle group including the needle inserted into the skin among the plurality of needle groups on the basis of the skin impedance measured by the skin impedance measurement unit, and to transmit the RF energy to the sensed needle group.

A method of controlling an RF treatment device according to an embodiment of the present disclosure is a method of controlling an RF treatment device including a body having an RF generator and a handpiece having a rolling member having a plurality of needles formed to protrude, the method including detecting information related to a speed of the rolling member that slides on a surface of skin, and controlling output of the RF energy transmitted to the plurality of needles from the RF generator on the basis of the detected information.

In the controlling the output of the RF energy, the output of the RF energy may be increased when the detected information corresponds to a state where the speed of the rolling member is higher than a reference speed.

In the controlling the output of the RF energy, the output of the RF energy may be decreased when the detected information corresponds to a state where the speed of the rolling member is lower than the reference speed.

The method may further include measuring impedance of the skin on which the rolling member slides. In the controlling the output of the RF energy, the output of the RF energy may be controlled on the basis of the detected information and the measured skin impedance.

In the measuring the skin impedance, the skin impedance may be measured on the basis of information about a current and a voltage transmitted through the needle inserted into the skin among the plurality of needles.

The information about the current and the voltage may be a current and a voltage generated by the RF energy transmitted to the needle inserted into the skin.

The method may further include measuring the impedance of the skin on which the rolling member slides, sensing a needle inserted into the skin among the plurality of needles on the basis of the measured skin impedance, and controlling the rolling member to transmit the RF energy to the sensed needle inserted into the skin.

The rolling member may be configured such that the plurality of needles is divided into a plurality of needle groups, and the RF energy is independently transmitted to each needle group. In the controlling the rolling member, the rolling member may be configured to sense a needle group including the sensed needle inserted into the skin among the plurality of needle groups on the basis of the skin impedance measured by the skin impedance measurement unit, and to transmit the RF energy to the sensed needle group.

The speed of the rolling member may be a rotating speed or a moving speed of the rolling member.

1. An RF treatment device comprising:
a body having an RF generator, and a handpiece connected to the body,
wherein the handpiece comprises:
a rolling member having a plurality of needles formed to protrude; and
a body part rotatably supporting the rolling member while at least some of the plurality of needles are exposed, transmitting, to the rolling member, RF energy transmitted from the RF generator, and formed so that the RF energy is transmitted to the rolling member even while the rolling member is rotating; and
wherein the RF treatment device further comprises:
a speed measurement unit for detecting information related to a speed of the rolling member; and
an RF control unit for controlling output of the RF energy on the basis of information detected by the speed measurement unit.

2. The RF treatment device of claim 1, wherein the RF control unit increases the output of the RF energy when information detected by the speed measurement unit corresponds to a state where the speed of the rolling member is higher than a reference speed.

3. The RF treatment device of claim 1, wherein the RF control unit decreases the output of the RF energy when information detected by the speed measurement unit corresponds to a state where the speed of the rolling member is lower than the reference speed.

4. The RF treatment device of claim 1, further comprising:
a skin impedance measurement unit configured to measure impedance of skin on the basis of information about the RF energy transmitted through the needle inserted into the skin among the plurality of needles.

5. The RF treatment device of claim 4, wherein the RF control unit controls the output of the RF energy on the basis of the skin impedance measured by the skin impedance measurement unit.

6. The RF treatment device of claim 1, further comprising:
a skin impedance measurement unit configured to measure the skin impedance on the basis of information about a current and a voltage transmitted through the needle inserted into the skin among the plurality of needles,
wherein the rolling member is configured such that the plurality of needles is divided into a plurality of needle groups, and the RF energy transmitted through the body part is independently transmitted to each needle group,
whereby the rolling member is configured to sense a needle group including the needle inserted into the skin among the plurality of needle groups on the basis of the skin impedance measured by the skin impedance measurement unit, and to transmit the RF energy to the sensed needle group.

7. A handpiece connected to a body having an RF generator, the handpiece comprising:
a rolling member having a plurality of needles formed to protrude;
a body part rotatably supporting the rolling member while at least some of the plurality of needles are exposed, transmitting, to the rolling member, RF energy transmitted from the RF generator, and formed so that the RF energy is transmitted to the rolling member even while the rolling member is rotating; and
a speed measurement unit for detecting information related to a speed of the rolling member.

8. The handpiece of claim 7, further comprising:
an RF control unit for controlling output of the RF energy on the basis of information detected by the speed measurement unit.

9. The handpiece of claim 7, wherein the speed of the rolling member is a rotating speed or a moving speed of the rolling member.

10. The handpiece of claim 7, further comprising:
a skin impedance measurement unit configured to measure the skin impedance on the basis of information about a current and a voltage transmitted through the needle inserted into the skin among the plurality of needles.

11. The handpiece of claim 10, wherein the rolling member is configured such that the plurality of needles is divided into a plurality of needle groups, and the RF energy transmitted through the body part is independently transmitted to each needle group,
whereby the rolling member is configured to sense a needle group including the needle inserted into the skin among the plurality of needle groups on the basis of the skin impedance measured by the skin impedance measurement unit, and to transmit the RF energy to the sensed needle group.

12. A method of controlling an RF treatment device comprising a body having an RF generator, and a handpiece having a rolling member having a plurality of needles formed to protrude, the method comprising:
detecting information related to a speed of the rolling member that slides on a surface of skin; and
controlling output of the RF energy transmitted to the plurality of needles from the RF generator on the basis of the detected information.

13. The method of claim 12, wherein, in the controlling the output of the RF energy, the output of the RF energy is increased when the detected information corresponds to a state where the speed of the rolling member is higher than a reference speed.

14. The method of claim 12, wherein, in the controlling the output of the RF energy, the output of the RF energy is decreased when the detected information corresponds to a state where the speed of the rolling member is lower than the reference speed.

15. The method of claim 12, further comprising:
measuring impedance of the skin on which the rolling member slides,
wherein, in the controlling the output of the RF energy, the output of the RF energy is controlled on the basis of the detected information and the measured skin impedance.

16. The method of claim 15, wherein, in the measuring the skin impedance, the skin impedance is measured on the basis of information about a current and a voltage transmitted through the needle inserted into the skin among the plurality of needles.

17. The method of claim 16, wherein the information about the current and the voltage is a current and a voltage generated by the RF energy transmitted to the needle inserted into the skin.

18. The method of claim 12, further comprising:
measuring the impedance of the skin on which the rolling member slides;
sensing a needle inserted into the skin among the plurality of needles on the basis of the measured skin impedance; and
controlling the rolling member to transmit the RF energy to the sensed needle inserted into the skin.

19. The method of claim 18, wherein the rolling member is configured such that the plurality of needles is divided into a plurality of needle groups, and the RF energy is independently transmitted to each needle group, and
wherein, in the controlling the rolling member, the rolling member is configured to sense a needle group including the sensed needle inserted into the skin among the plurality of needle groups on the basis of the skin impedance measured by the skin impedance measurement unit, and to transmit the RF energy to the sensed needle group.

20. The method of claim 12, wherein the speed of the rolling member is a rotating speed or a moving speed of the rolling member.
